(19) United States
(12) Patent Application Publication
(10) Pub. No.: US 2019/0298720 A1
(43) Pub. Date: Oct. 3, 2019

(54) USE OF SRC FAMILY KINASE INHIBITOR IN RIBOSOMAL DISORDER

(71) Applicant: BIOXCEL CORPORATION, Branford, CT (US)

(72) Inventor: Krishnan NANDABALAN, Branford, CT (US)

(21) Appl. No.: 16/307,579

(22) PCT Filed: Jun. 9, 2017
(86) PCT No.: PCT/US2017/036769

§ 371 Date: Dec. 6, 2018

Related U.S. Application Data
(60) Provisional application No. 62/347,671, filed on Jun. 9, 2016.

(51) Int. Cl. A61K 31/517; A61P 7/06

(52) U.S. Cl. CPC A61K 31/517; A61P 7/06

(57) ABSTRACT

The present disclosure provides methods and compositions for the use of a Src family kinase (SFK) inhibitor for the treatment of anemia associated with ribosomal disorders, such as diamond blackfan anemia.

### FIELD OF THE INVENTION

The present disclosure relates to methods of increasing or stimulating erythropoiesis. Specifically, the present disclosure relates to methods of treating anemia associated with ribosomal disorders e.g. diamond blackfan anemia. More specifically, the present disclosure relates to methods or compositions for treating diamond blackfan anemia (DBA) by administering a Src family kinase (SFK) inhibitor.

### CROSS-REFERENCE TO RELATED APPLICATION

This application claims priority to U.S. Provisional Application Ser. No. U.S. 62/347,671 filed on Jun. 9, 2016, the disclosure of which is herein incorporated by reference in its entirety for all purposes.

### BACKGROUND

Src is the prototype of a family of protein tyrosine kinases and was discovered as the transforming factor arising from avian Rous sarcoma virus (hence “Src”). Src Family Kinases (SFKs), that include the ubiquitously expressed family members pp60c˜Src tyrosine kinase (c-Src), c-Yes and c-Fyn, are protein tyrosine kinases that play important roles in modulating intracellular signal transduction pathways in cells in response to a variety of extracellular and intracellular stimuli. The activities of Src are frequently dysregulated and are increased in human cancers, leading to aberrant signal transduction linked to disease progression.

It is further known that the predominant role of c-Src tyrosine kinase is to regulate the assembly of focal adhesion complexes through interacting with a number of cytoplasmic proteins including, for example, focal adhesion kinases and paxillin. In addition, c-Src tyrosine kinase is coupled to signalling pathways that regulate the actin cytoskeleton which facilitates cell motility. Likewise, c-Src, c-Yes and c-Fyn tyrosine kinases play important roles in integrin mediated signalling and in disrupting cadherin dependent cell-cell junctions (Owens et al, Molecular Biology of the Cell, 2000, 11, 51-64 and Klinghoffer et al, EMBO Journal, 1999, 18, 2459-2471).

It is also known that c-Src tyrosine kinase enzyme is involved in controlling osteoclast-driven bone resorption (Soriano et. al, Cell, 1991, 64, 693-702; Boyce et. al, J. Clin. Invest., 1992, 90, 1622-1627; Yoneda et al, J. Clin. Invest., 1993, 91, 2791-2795 and Missbach et al, Bone, 1999, 24, 437-49).

Oliver Gautschi et al. (The Journal of Cancer Research in Vol. 68 (7), 2008, 2250-2258) discloses that a SFK inhibitor, AZD0530, downregulates the Id1 expression in lung cancer cell lines by interacting with bone morphogenetic protein 2 (BMP)-Smad-Id pathways. However, this article primarily focuses on Id1 expression in cancerous cells and its suppression by the administration of AZD0530 while the present disclosure relates to the regulation of Id2 expression by administering the SFK inhibitor, preferably AZD0530.

The present disclosure has discovered that the inhibition of the activities of the Src family tyrosine kinase(s) is particularly useful for treating anemia associated with ribosomal disorders. SFK inhibitors inhibit Lyn kinase and thereby regulate the levels of Id2 proteins and result in restoring the normal functioning of GATA1, which eventually binds to SCL and E2A proteins and leads to stimulate erythropoiesis, thereby maintaining the optimum level of haemoglobin and red blood corpuscles in such patients.

Among such disorders, DBA is one such ribosomal disorder and it is a rare genetic blood disorder of bone marrow that the DBA patients are unable to produce enough red blood cells. It is also known as blackfan-diamond anemia, inherited red cell aplasia, inherited erythroblastopenia, or chronic congenital erythrogenesis imperfecta. It is also categorized as one of the emerging group of disorders known as ribosomopathies or ribosomal disorder. DBA affects around 5 to 7 per millions of infants worldwide and it is usually diagnosed before 12 months of age. DBA patients have mutations in the ribosomal protein genes. Genes susceptible for the mutations in DBA patients are RPL3, RPL5, RPL11, RPL15, RPL26, RPL31, RPL35A, RPL36, RPS7, RPS10, RPS14, RPS17, RPS19, RPS24, RPS26 and RPS29 gene. Approximately 20-25% patients with DBA may be identified with mutation in RPS19 gene.

Min-Ying Zhang et al. (Blood, 1997 (American Society of Hematology), Vol. 90 (5), Page No. 2068-2074) discloses that Id2 protein is highly expressed in DBA patients and it leads to late erythroid differentiation. Wonil Kim et al. (Blood, 2014, Vol. 124 (10), Page No. 1586-1596) discloses that the reduction of Id2 expression increased the expression of GATA1, Eklf and EpoR, which are required for proper erythropoiesis. Huajie Li et al. (Blood, 2010, Vol. 116 (7), Page No. 1060-1069) discloses transcriptional growth factor independence 1 (Gfi1) and B cell transcriptional network repress Id2 expression and it is required for B-cell and myeloid development. These references collectively suggest that Id2 protein is overexpressed in DBA patients and thus regulation of Id2 protein is required for proper erythropoiesis.

US Publication No. 20150265627 discloses the use of calmodulin inhibitor for treating ribosomal disorder such as DBA. However, the present disclosure highlights the regulation of Id2 protein by administering a SFK inhibitor for the treatment of DBA.

In addition, the treatment of DBA mainly includes corticosteroid therapy, blood transfusion, stem cell transplant therapy and iron chelation therapy (deferasirox and deferoxamine). These therapies have their own limitations and/or side effects. Corticosteroid therapy has unwanted profound effects on linear growth as well as physical and neurocognitive development in infants. Transfusion therapy requires appropriate blood supply and sometimes blood transfusion may lead to immunosuppression, which may increase the risk of infection or cancer recurrence. Stem cell transplantation therapy involves replacement of disease forming cells with another person's healthy cells and the success depends only on the close match between the child and the donor's age. Deferasirox causes gastrointestinal side effects such as gastrointestinal haemorrhage, and renal or hepatic failures. Deferoxamine causes reddish coloured urine, diarrhoea, seizure, blurred vision etc. Currently, all the available therapies for the treatment of DBA patients are associated with several limitations and/or side effects. Hence, there is a high unmet need for an effective therapy with less severe side effects which can increase the survival rates in the DBA patients.

The present disclosure suggests that the targeted inhibition of SFKs would rectify the disease mechanisms and correct the arrested erythropoiesis as SFK inhibitor(s) target the pathophysiology of the disease. The present disclosure is based on inventor's findings that the subjects having a ribosomal disorder that is associated with RPS19 mutations have high levels of Id2 proteins, which can negatively regulate GATA1, SCL and E2A proteins binding to promoter and therefore, can inhibit the erythroid differentiation and maturation processes, namely, the erythropoiesis. Therefore, the inventors of the present disclosure surprisingly found that inhibiting Lyn kinase can regulate the expression of Id2 protein and therefore, restoring the erythropoietic defects in DBA patients (i.e., improvement in anemic conditions). The present therapies can be a safe and an effective therapeutic intervention for DBA patients.

### SUMMARY OF THE INVENTION

The present disclosure provides methods of treating a subject suffering from anemia associated with ribosomal disorders comprising administering to said subject effective amounts of SFK inhibitors.

The present disclosure also provides pharmaceutical compositions for use in methods of treating a subject suffering from anemia associated with ribosomal disorders comprising administering to said subject the pharmaceutical compositions comprising effective amounts of a SFK inhibitor.

Accordingly, one aspect of the present disclosure relates to methods of increasing, stimulating, or promoting erythropoiesis in a subject comprising administrating an effective amount of a SFK inhibitor.

In all aspects of the present disclosure, a SFK inhibitor includes, but is not limited to Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326. The preferred SFK inhibitor is Saracatinib, Ponatinib, Bosutinib, Rebastinib, Bafetinib and Dasatinib. In one aspect, the SFK inhibitor is Saracatinib [AZD0530].

In one aspect, the present disclosure is directed to methods and compositions for treating DBA such as DBA1, DBA2, DBA3, DBA4, DBA5, DBA6, DBA7, DBA8, DBA9, DBA10, DBA11, DBA12 or DBA13. In one aspect, the DBA is DBA1.

In some aspects, a subject with ribosomal disorders has a mutation in ribosomal protein 19 (RPS19). In alternative embodiments, a subject with ribosomal disorders has a mutation in ribosomal protein from at least one of, but not limited to RPL3, RPL5, RPL11, RPL15, RPL26, RPL31, RPL35A, RPL36, RPS7, RPS10, RPS14, RPS17, RPS19, RPS24, RPS26 or RPS29 gene. In one aspect, a subject with ribosomal disorders has a mutation in RPS19.

In some aspects, the present disclosure encompasses methods for treating a subject suffering from anemia associated with ribosomal disorders e.g. DBA or inherited erythroblastopenia comprising administrating an effective amount of a SFK inhibitor e.g. Saracatinib (AZD0530), wherein the subject has DBA1, DBA2, DBA3, DBA4, DBA5, DBA6, DBA7, DBA8, DBA9, DBA10, DBA11, DBA12 or DBA13. In one aspect, the subject has DBA1.

In some aspects, the methods encompass treating a subject having DBA, which comprise administering an effective amount of Saracatinib to the subject that elevates the levels of GATA1, E2A and/or SCL. In some aspects, administering Saracatinib to the subject elevates GATA1 for normal erythrocyte development, wherein the DBA is associated with RPS19 mutation,

In one aspect, the present disclosure provides methods of treating a subject having DBA that comprise administering to said subject effective amounts of Saracatinib wherein the DBA is associated with RPS19 mutation.

In one aspect, the present disclosure provides pharmaceutical compositions for use in methods of treating a subject having DBA that comprises administering to said subject the pharmaceutical compositions comprising effective amounts of Saracatinib, and wherein the diamond blackfan anemia is associated with RPS19 mutation.

In one aspect, the present disclosure provides pharmaceutical compositions for use in methods of treating a subject having diamond blackfan anemia, wherein the methods comprise administering to said subject the pharmaceutical compositions comprising effective amounts of Saracatinib. In another aspect, the effective amounts include the effective daily doses administered to the subject in between about 5 mg to about 250 mg, about 50 mg to about 250 mg, and about 100 mg to about 200 mg.

In one aspect, the methods encompass treating a subject having diamond blackfan anemia. In one aspect, the methods comprise administering effective amounts of Saracatinib, wherein the daily dose of Saracatinib ranges from about 0.5 mg to about 500 mg, about 5 mg to about 250 mg, about 50 mg to about 250 mg. In one aspect, the daily dose of Saracatinib is about 100 mg to about 250 mg. In another aspect, the daily dose of Saracatinib is about 100 mg to about 200 mg.

In another aspect, the present disclosure also provides kits that comprise compositions containing SFK inhibitors as disclosed herein for the use in the methods for treating a subject suffering from anemia associated with ribosomal disorders as disclosed herein.

### BRIEF DESCRIPTIONS OF DRAWINGS

FIG. 1 depicts the effect of Saracatinib (25 μM, 50 μM, and 100 μM) on improving the erythropoietic defects produced by RPS19 morpholino (1 ng) in zebrafish model of DBA. 0-Dianisidine staining was used to stain the embryos and reflect the concentration of haemoglobin present in the zebrafish. The treatment groups are: (i) Control Morpholino, Egg water; (ii) RPS19 Morpholino, Egg water; and (iii) RPS19 Morpholino+Saracatinib injected 1 cell stage and aqueous exposure. Respective controls have been represented with respect to Saracatinib treatment, at each concentration of Saracatinib tested in the zebrafish.

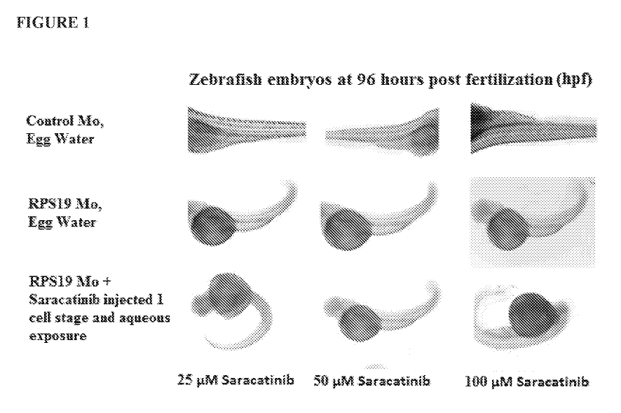

### DETAILED DESCRIPTION OF THE INVENTION

Abbreviations:

CD: Cluster of differentiation

### DBA: Diamond Blackfan Anemia

DMSO: Dimethyl sulfoxide

### EPO: Erythropoietin

E2A: Transcription factor E2 Alpha
Gfi1: Growth factor independence 1
GATA1: Erythroid transcription factor/GATA binding factor 1
HSC: hematopoietic stem cells
Hpf: Hours post fertilization

### Id2: Inhibitor of DNA Binding 2

MCV: Mean corpuscular volume
MCHC: Mean corpuscular haemoglobin concentration
PBS: Phosphate buffered saline

### PFA: Paraformaldehye

RBC: Red blood cells

### RP: Ribosomal Protein

SFK: Src family kinase(s)
SCF: Stem cell factor
SCL: Stem cell leukemia gene

In the normal adult erythropoiesis process, RBCs or erythrocytes are formed by the hematopoietic stem cells in response to a specific stimulus or signal. In hematopoietic progenitors, these signals are transduced to transcription factors like GATA-1, E2A, SCL in the nucleus, and the progenitor cells differentiate and mature into erythrocytes by changing their gene expression profiles. This erythroid cell differentiation and maturation from hematopoietic stem cells (HSCs) occurs through several steps which are highly regulated by a family of Id2 proteins called DNA binding proteins 2. DNA binding proteins 2 play an important role in regulating erythropoiesis in a dynamic process. The levels of these proteins are very low when the normal erythropoietic process has to occur. However, the levels increase when the process has completed. The Id2 proteins are reported as negative regulator of GATA1, which is central to erythropoietic process. More importantly, the Id2 proteins are indirectly regulated by SFKs.

Accordingly, the inventor of the present disclosure demonstrates herein that the SFK pathway is important in erythropoiesis, and small molecules that inhibit SFK may be effective therapies for patients with anemia associated with RPS19 mutation, specifically DBA. Therefore, a SFK inhibitor can inhibit Lyn kinase activity in a ribosomal protein knockdown (i.e., the DBA patient). This lead to subsequent regulation of Id2 proteins, followed by induction of GATA1 along with SCL and E2A protein expression in the progenitor cells to a normal erythrocyte development.

Therefore, the SFK inhibitor(s) disclosed herein can be used in methods for treating a subject suffering from anemia associated with ribosomal disorder including diamond blackfan anemia (DBA) such as DBA1, DBA2, DBA3, DBA4, DBA5, DBA6, DBA7, DBA8, DBA9, DBA10, DBA11, DBA12 or DBA13 in a human subject.

The details of the present disclosure are as follows:

### Definitions

The terms disclosed herein are to be given their ordinary meaning in the art unless otherwise indicated. Other specifically defined terms are to be construed in a manner consistent with the definition provided herein. The definitions are provided to aid in describing particular embodiments, and are not intended to limit the claimed disclosure. Unless otherwise defined, all technical and scientific terms used herein have the same meaning as commonly understood by one of ordinary person skill in the art.

The term “ribosomal disorder” or “ribosomal protein disorder” refers to a disease or a disorder linked to a mutated and/or abnormal function of a ribosome protein. It can include a disease due to mutation in a ribosomal protein, or a disease due to a decreased level, or partial loss of function, of a ribosomal protein, or alternatively, a disease due to an increased level of a ribosomal protein, as compared to a normal healthy control subject. The term ribosomal disorder includes genetic diseases of ribosomal proteins, which include but are not limited to DBA.

The term “ribosomopathy” or “ribosomopathies” refers to any disease or malfunction of ribosomes. Ribosomes are small organelles found in all cells which are involved in the production of proteins by translating messenger RNA. A disease or malfunction of ribosomes include, but are not limited to (i) diseases of ribosomal biogenesis proteins, (ii) diseases of small nucleolar ribonuceloproteins, and (iii) diseases of ribosomal proteins, which are reviewed in Freed et al., Mol. Biosyst. 2010; 6(3); 481-493 entitled “When ribosomes go bad: diseases of ribosome biogenesis”, which is incorporated herein in its entirety by reference. Diseases of ribosomal biogenesis proteins include, but are not limited to DBA.

The terms “treat”, “treatment” or “treating,” which can be used interchangeably with respect to treatment of a disease or a disorder, refers to preventing the development of the disease, altering the course of the disease (for example, slowing the progression of the disease). It can refer to reversing or reducing one or more symptoms of the disease and/or reducing one or more biochemical markers in a subject. It can also refer to preventing one or more symptoms from worsening or progressing, promoting recovery or improving prognosis in a subject who is at risk of the disease, as well as slowing or reducing progression of existing disease. The term “treating” encompasses reducing or alleviating at least one adverse effect or symptom of anemia associated with inappropriate ribosomal protein function. As used herein with respect to a ribosomal protein disorder, the term “treating” is used to refer to reducing a symptom and/or a biochemical marker of a ribosomal protein disorder by at least 10%. For example, an increase in GATA1 level in CD34+ cells in the subject, at least 10% decrease in Lyn kinase activity or a return of haemoglobin back to normal levels, or a restoration or prevention of craniofacial deformities or increase in the erythroid proliferation or increase in the mean corpuscular haemoglobin concentration. For example, but are not limited to, an increase in levels of GATA1 in CD34+ cells in the subject, as an illustrative example only, by 10%, would be considered effective treatments by the methods as disclosed herein.

A “pharmaceutical composition” refers to a chemical or biological composition suitable for administration to a mammalian subject. Such compositions may be specifically formulated for administration via one or more of a number of routes, which include but are not limited to, oral, parenteral, intravenous, intraarterial, subcutaneous, intranasal, sublingual, intraspinal, intracerebroventricular, and the like.

The term “effective amount” is used interchangeably with the term “therapeutically effective amount” and refers to the amount of at least one agent, e.g., SFK inhibitor of a pharmaceutical composition, at dosages and for periods of time necessary to achieve the desired therapeutic result, for example, to reduce or stop at least one symptom of anemia associated with ribosomal disorder or ribosomopathy such as a symptom of high levels of Id2 proteins in CD34+ cells in the subject. For example, an effective amount using the methods as disclosed herein would be considered as the amount sufficient to reduce a symptom of anemia associated with ribosomal disorder or ribosomopathy by at least 10%. An effective amount as used herein would also include an amount sufficient to prevent or delay the development of a symptom of the disease, alter the course of a symptom disease (for example but not limited to, slow the progression of a symptom of the disease), or reverse a symptom of the disease. Accordingly, the term “effective amount” or “therapeutically effective amount” as used herein refers to the amount of therapeutic agent (e.g. at least one SFK inhibitor as disclosed herein) of pharmaceutical composition to alleviate at least one symptom of anemia associated with ribosomal disorder or ribosomopathy, e.g. DBA. In other words, “therapeutically effective amount” of a SFK inhibitor as disclosed herein is the amount of a SFK inhibitor which exerts a beneficial effect on, for example, the symptoms of anemia associated with ribosomal disorder or ribosomopathy. The dosage administered, as single or multiple doses, to an individual will vary depending upon a variety of factors, including pharmacokinetic properties of the SFK inhibitor, the route of administration, conditions and characteristics (sex, age, body weight, health, size) of subjects, extent of symptoms, concurrent treatments, frequency of treatment and the effect desired. A therapeutically effective amount is also one in which any toxic or detrimental effects of the therapeutic agent are outweighed by the therapeutically beneficial effects. In general, the phrases “therapeutically-effective” and “effective for the treatment, prevention, or inhibition” are intended to qualify a SFK inhibitor as disclosed herein which will achieve the goal of reduction in the severity of at least one symptom of anemia associated with ribosomal protein disease or ribosomopathy.

The term “Src family kinase inhibitor” or “SFK inhibitor” refers to any chemical compound, which inhibits one or more members of the SFK, which includes, but is not limited to Src, Fyn, Yes, Lck, Lyn, Hck, Fgr, Frk and Blk, preferably Lyn. Examples of a SFK inhibitor include but are not limited to Saracatinib [AZD0530], Quercetin, Bosutinib, Dasatinib, Rebastinib, Staurosporine, A419259, KX2-391, PP1, PP2, Ponatinib, SU6657, Bafetinib, USC15A, CGP77675, CGP76030, SI35, SI40, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018, pD166326 and their pharmaceutically acceptable salt, solvate, derivative, prodrug or analogue thereof. The preferred SFK inhibitor is Saracatinib [AZD0530], Ponatinib, Bosutinib, Rebastinib, Bafetinib and Dasatinib. More preferably, SFK inhibitor is Saracatinib [AZD0530].

The word “Src family kinase inhibitor” includes Src family kinase inhibitor as a base or its pharmaceutically acceptable salt, solvate, derivative, pro-drug or analogue thereof.

The term “Saracatinib” includes Saracatinib base as a base or its pharmaceutically acceptable salt, solvate, derivative, pro-drug or analogue thereof. The term “Saracatinib” is also referred as AZD0530 or “N-(5-chloro-1,3-benzodioxol-4-yl)-7-[2-(4-methyl-1-piperazinyl) ethoxy]-5-[(tetrahydro-2H-pyran-4-yl)oxy]-4-quinazolinamine”.

As used herein, the term “about” refers to an amount somewhat more or less than the stated parameter value, for example plus or minus five or ten percent of the object that “about” modifies, or as one of skill in the art would recognize from the context. The term “about” also includes the value referenced.

The term “subject” herein refers to human and other mammalian subjects that receive either prophylactic or therapeutic treatments. The terms “subject,” “patient,” and “individual” are used interchangeably. The other mammalian subject in the present disclosure include mammals such as mice, rats, sheep, rabbits, dogs, cats, cows, pigs and non-human primates.

### 1. SRC FAMILY KINASE INHIBITOR(S) (SFK INHIBITOR(S))

The present disclosure relates to methods and compositions to inhibit SFKs. In some embodiments, the SFK inhibitor as disclosed herein can be used to inhibit the cellular SFK. In some embodiments, the SFK inhibitor is an inhibitor of all the genes (Src, Fyn, Yes, Lck, Lyn, Hck, Fgr, Frk and Blk), preferably Lyn kinase. For example, the term SFK inhibitor encompasses an inhibitor of the SFK, an inhibitor of the Src kinase, an inhibitor of the Lyn kinase, an inhibitor of the Fyn kinase, an inhibitor of the Yes kinase, an inhibitor of the Lck kinase, an inhibitor of the Hck kinase, an inhibitor of the Fgr kinase, an inhibitor of the Frk kinase, or an inhibitor of the Blk kinase. In some embodiments, the SFK inhibitor inhibits preferably Lyn kinase and/or Src kinase.

The inhibitor of SFK includes but is not limited to Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326. Preferably the Src family kinase inhibitor is Saracatinib [AZD0530], Ponatinib, Bosutinib, Rebastinib, Bafetinib and Dasatinib. More preferably, the Src family kinase inhibitor is Saracatinib [AZD0530].

### 2. METHOD OF USES

In some embodiments, the present disclosure provides methods of treating, preventing or ameliorating anemia associated with ribosomal disorder in subjects comprising administering an effective amount of SFK inhibitor, thereby treating said anemia associated with ribosomal disorder.

The ribosomal disease or disorder is DBA or inherited erythroblastopenia such as DBA1, DBA2, DBA3, DBA4, DBA5, DBA6, DBA7, DBA8, DBA9, DBA10, DBA11, DBA12 or DBA13. In one embodiment, the DBA or inherited erythroblastopenia is DBA1.

In some embodiments, a SFK inhibitor as disclosed herein can be used to treat anemia associated with ribosomal disorder. For instance, the SFK inhibitors can be used to treat a subject who has a mutation in one or more ribosomal proteins, or have a decreased level of the ribosomal protein such as RPL3, RPL5, RPL11, RPL15, RPL26, RPL31, RPL35A, RPL36, RPS7, RPS10, RPS14, RPS17, RPS19, RPS24, RPS26 and/or RPS29, preferably RPS19. In one embodiment, the inhibitor of SFK is Saracatinib and the ribosomal disorder is DBA. In one embodiment, the ribosomal disorder is DBA1. Without being bound by theory, a mutation or variant in RPS19 causes DBA1, a mutation or variant in RPS24 causes DBA3, a mutation or variant in RPS17 causes DBA4, a mutation or variant in RPL35A causes DBA5, a mutation or variant in RPL5 causes DBA6, a mutation or variant in RPL11 causes DBA7, a mutation or variant in RPS7 causes DBA8, a mutation or variant in RPS10 causes DBA9, a mutation or variant in RPS26 causes DBA10, a mutation or variant in RPL26 causes DBA11, a mutation or variant in RPL15 causes DBA12, and a mutation or variant in RPS29 causes DBA13.

Accordingly, in some embodiments, the SFK inhibitor(s) as disclosed herein is used in a method of treating disorders associated with mutation in ribosomal protein 19 and/or 24 (RPS19 and/or RPS24). The phenotype of DBA patients indicates a haematological stem cell defect specifically affecting the erythroid progenitor population.

In some embodiments, Saracatinib can rescue the erythropoietic defect and increase hemoglobin concentrations in the RPS19 knock-down zebrafish embryos. In one embodiment, Saracatinib is suitable for the treatment of DBA.

In some embodiments, a SFK inhibitor is administered to the subject suffering from DBA to produce the following effects: regulate the level of Id2 expression, increase GATA-1 levels, decrease Lyn kinase activity that leads to the increase in haemoglobin level in the subject or the increase in the erythroid proliferation.

In some embodiments, the present disclosure relates to method of increasing or stimulating or promoting erythropoiesis in a subject by administering therapeutically effective amounts of a SFK inhibitor (e.g. Saracatinib) as disclosed herein.

In some embodiments, the methods and the SFK inhibitor as disclosed herein can be used to treat a subject with a ribosomal disorder, such as DBA, who has a symptom of macrocytic anemia and/or craniofacial abnormalities.

In an embodiment, the SFK inhibitor is Saracatinib.

In some embodiments, the methods further comprise administering another therapeutic agent to treat the ribosomal disorder. In some embodiments, the therapeutic agents are selected from the group consisting of: corticosteroids, calmodulin inhibitor, blood transfusions and bone marrow transplants and other treatments known to persons of ordinary skill in the art. Corticosteroids can be used to treat anemia in DBA. Blood transfusions can also be used to treat severe anemia in DBA. Periods of remission may occur, during which transfusions and steroid treatments are not required.

### 3. SELECTION OF SUBJECTS FOR ADMINISTRATION WITH A PHARMACEUTICAL COMPOSITION

In some embodiments, a subject amenable or suitable for treatment with a composition comprising a SFK inhibitor as disclosed herein can be selected based on decreased levels of haemoglobin, decreased erythroid proliferation and increased levels of Id2 expression in CD34+ cells, as compared to a control normal levels of haemoglobin and Id2 expression level in a normal subject. Additionally, a subject amenable or suitable for treatment with a composition comprising a SFK inhibitor as disclosed herein can be selected based on decreased levels of GATA-1 expression/increased activity of Lyn kinase in CD34+ cells as compared to a control GATA-1 expression level/Lyn kinase activity. In some embodiments, the normal levels are based on the level of Id2 expression, GATA1 expression levels, Lyn activity in a sample from a normal subject without a ribosomal disorder, a control cell line, or cells from a normal tissue sample.

In some embodiments, the levels of haemoglobin, erythroid proliferation, Id2 expression, GATA1 expression levels are measured in a biological sample comprising hematopoietic cells or erythroid cells or erythroid differentiated cells. In some embodiments, a biological sample obtained from the subject comprises blood cells, or can contain serum plasma, blood or tissue samples. In one embodiment, the biological sample includes, but is not limited to blood, plasma, serum, urine, spinal fluid, plural fluid, nipple aspirates, lymph fluid, external secretions of the skin, respiratory, internal and genitourinary tracts, bile, tears, sweat, saliva, organs, milk cells and primary ascites cells, biopsy tissue sample, an in vitro or ex vivo cultivated biopsy tissue sample.

Typically, a diagnosis of DBA is made through a blood count. Decreased or the absence of red blood cell precursors in the bone marrow biopsy can indicate the presence of DBA. A diagnosis of DBA is made on the basis of anemia, pure red cell aplasia, low reticulocyte (immature red blood cells) counts, macrocytic anemia, reticulocytopenia, and diminished erythroid precursors in bone marrow. Features that support a diagnosis of DBA includes the presence of congenital abnormalities, macrocytosis, mean corpuscular volume (MCV), elevated foetal haemoglobin, and elevated adenosine deaminase levels in red blood cells. About 20-25% of DBA patients may be identified with a genetic test for mutations in the RPS19 gene. Approximately 10-25% of DBA cases have a family history of disease, and most pedigrees suggest an autosomal dominant mode of inheritance.

### 4. PHARMACEUTICAL COMPOSITIONS

Another aspect of the present disclosure relates to pharmaceutical compositions for use in the treatment of anemia associated with ribosomal disorder e.g., DBA. In some embodiments, a pharmaceutical composition of the disclosure comprises an effective amount of at least one SFK inhibitor as disclosed herein.

In some embodiments, a composition of the present disclosure comprises a SFK inhibitor as disclosed herein and is formulated for ribosomal disorder such as DBA.

In therapeutic applications in the present disclosure, an effective amount or effective dose of a pharmaceutical composition comprises a SFK inhibitor as disclosed herein that can be administered to the subject suffering from DBA.

A SFK inhibitor as disclosed herein can be administered therapeutically to a subject prior to, simultaneously or sequentially with other therapeutic regimens or agents (e.g. multiple drug regimens), in a therapeutically effective amount. In some embodiments, a SFK inhibitor administered concurrently with other therapeutic agents can be administered in the same or different compositions. Additional therapeutic agents or regimens include, but are not limited to, steroids, corticosteroids, calmodulin inhibitors, blood transfusions and bone marrow transplants.

Depending on the routes of administration, an effective dosage of a SFK inhibitor (e.g. Saracatinib) disclosed herein to a subject such as a human subject can be determined through the pharmacokinetics and bioavailability of the SFK inhibitor (e.g. Saracatinib). In some embodiments, an effective dosage of a SFK inhibitor can also be determined by analyzing the dose-response relationship specific to a SFK inhibitor in a mouse animal model.

The therapeutically effective dose can be determined by, using cell culture assays or animal studies. An effective dose of a SFK inhibitor (e.g. Saracatinib) can be determined in an animal model by measuring the levels of haemoglobin or red blood cells number over the course of treatment with a SFK inhibitor as compared to its cohorts without the treatment. In some embodiments, a dosage comprising a SFK inhibitor is considered to be effective if the pharmaceutical composition increases haemoglobin levels, red blood cell number, and/or increases the levels of GATA1 in CD34+ cells by at least about 15%, at least about 20%, at least about 30%, at least about 40%, at least about 50%, at least about 60%, at least about 70%, at least about 80%, at least about 90%, at least about 95%, at least about 99%, at least about 100%, inclusive of all ranges and subranges therebetween, compared to a control (e.g. in the absence of a SFK inhibitor).

In some embodiments, a therapeutically effective amount of a SFK inhibitor administered to a subject is dependent upon factors including bioactivity and bioavailability of a SFK inhibitor (e.g. half-life and stability of a SFK inhibitor in the body), chemical properties of a SFK inhibitor (e.g. molecular weight, hydrophobicity and solubility); route and frequency of administration, time of administration (e.g. before or after a meal), and the like.

The active ingredients (e.g. a SFK inhibitor) of the pharmaceutical composition according to the present disclosure can be administered to an individual by any route known to persons skilled in the art. The routes of administration include, but are not limited to intradermal, transdermal (e.g. in slow release formulations), parenteral, intramuscular, intraperitoneal, intravenous, subcutaneous, oral, buccal, nasal, rectal, epidural, topical, intrathecal, rectal, intracranial, intratracheal and intranasal routes and the preferred route is parenteral or oral. Any other therapeutically efficacious route of administration can be used, for example, absorption through epithelial or endothelial tissues or systemic administration. In addition, a SFK inhibitor (e.g. Saracatinib) of the present disclosure can be administered together with pharmaceutically acceptable excipient(s) or adjuvant(s) such as pharmaceutically acceptable surfactants, excipients, carriers, diluents and vehicles.

Selection of pharmaceutically acceptable carriers (i.e. physiologically acceptable carriers) can depend on the routes of administration. For example, if the composition is orally administered, it can be formulated in coated tablets, liquids, caplets and so forth. Examples of liquid carriers can include, but are not limited to sterile aqueous solutions that contain no materials except for the active ingredients and water, or contain a buffer such as sodium phosphate at physiological pH value, physiological saline or both, such as phosphate-buffered saline. In some embodiments, aqueous carriers can contain more than one buffer salt, as well as salts such as sodium and potassium chlorides, dextrose, polyethylene glycol and other solutes. For topical application, the carrier may be in the form of, for example, and not by way of limitation, an ointment, cream, gel, paste, foam, aerosol, suppository, pad or gelled stick. In some embodiments, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. In one embodiment, Saracatinib is administered as an immediate release formulation. In one embodiment, Saracatinib is administered as an extended release formulation.

In some embodiments, a pharmaceutical composition as disclosed herein comprises a SFK inhibitor (e.g. Saracatinib) together with other therapeutics and a pharmaceutically acceptable excipient. Suitable carriers for a SFK inhibitor of the invention, and their formulations, are described in Remington's Pharmaceutical Sciences, 16th ed., 1980, Mack Publishing Co., edited by Oslo et al.

Oral formulations include excipients, such as pharmaceutical grades of mannitol, lactose, starch, magnesium stearate, sodium saccharine, cellulose, and magnesium carbonate. These compositions are in the form of solutions, suspensions, tablets, pills, capsules, sustained release formulations or powders and contain 10%-95% w/w of active ingredient, preferably 25%-70% w/w. In some embodiments, a SFK inhibitor as disclosed herein is often administered as pharmaceutical compositions comprising an active therapeutic agent, i.e. a SFK inhibitor, and a variety of other pharmaceutically acceptable excipients. See Remington's Pharmaceutical Science (16th ed., Mack Publishing Company, Easton, Pa., 1980). The compositions can also include, depending on the formulation, pharmaceutically-acceptable, non-toxic excipients, carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, non-therapeutic, non-immunogenic stabilizers and the like.

For parenteral (e.g. intravenous, subcutaneous, intramuscular) administration, a SFK inhibitor (e.g. Saracatinib) can be formulated as a solution, suspension, emulsion or lyophilized powder in association with a pharmaceutically acceptable parenteral vehicle (e.g. water, saline, dextrose solution, oils, saline, glycerol, or ethanol) and additives that maintain isotonicity (e g. mannitol) or chemical stability (e.g. preservatives and buffers). Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

In some embodiments, the route of administration is subcutaneous route. Intramuscular administration is another alternative route of administration. In some embodiments, a pharmaceutical composition comprising a SFK inhibitor can be administered as a formulation adapted for systemic delivery.

Alternatively, in some embodiments, a pharmaceutical composition can be incorporated into a gel, sponge, or other permeable matrix (e.g., formed as pellets or a disk). The composition comprising a SFK inhibitor (e.g. Saracatinib) can be administered in a single dose or in multiple doses, which are administered at different times. Further carriers include sustained release preparations such as semipermeable matrices of solid hydrophobic polymers, which matrices are in the form of shaped articles, e.g. liposomes, films or microparticles.

Additional formulations suitable for other modes of administration include intranasal and/or pulmonary formulations, suppositories, and transdermal applications. For suppositories, binders and carriers include, for example, polyalkylene glycols or triglycerides; such suppositories can be formed from mixtures containing the active ingredient in the range of about 0.5% to about 10%, preferably about 1% to about 2%.

In some embodiments, bioavailability of a SFK inhibitor (e.g. Saracatinib) according to the present disclosure can be also enhanced by encapsulating a SFK inhibitor in biocompatible delivery vehicles which increase the half-life of a SFK inhibitor in a human body. Exemplary biocompatible delivery vehicles include polymeric vehicles such as PEG-based vehicles, or liposome-based vehicles.

Topical application can result in transdermal or intradermal delivery. Other modes of administration include systemic delivery. In some embodiments, at least one SFK inhibitor (e.g. Saracatinib) as disclosed herein can be injected systemically such as by intravenous injection, or by injection or application to the relevant site, such as direct application to the site when the site is exposed in surgery. In some embodiments, a pharmaceutical composition of the present disclosure can be formulated in a tablet and used orally for systemic administration. In various embodiments, pharmaceutical compositions of the present disclosure can further comprise non-active ingredients (i.e. ingredients that have no therapeutic values for treatment of diseases, disorders or symptoms), such as physiologically acceptable carriers.

Bioabsorbable polymeric matrix suitable for delivery of a SFK inhibitor (e.g. Saracatinib) as disclosed herein, or variants or fragments or derivatives thereof can be selected from a variety of synthetic bioabsorbable polymers, which are described extensively in the literature. Such synthetic bioabsorbable, biocompatible polymers, which may release proteins over several weeks or months can include, for example, poly-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides).

In some embodiments, the present disclosure also provides compositions comprising a SFK inhibitor (e.g. Saracatinib) as discussed herein for practicing the therapeutic and prophylactic methods described herein. In some embodiments, combinations of a SFK inhibitor (e.g. Saracatinib) and another therapeutic agent can be tailored to be combined in a pharmaceutical composition, where each therapeutic can target a different symptom, a different disease or a different disorder. In further embodiments, a SFK inhibitor and another therapeutic can be mixed together in a pharmaceutical composition as disclosed herein. In other embodiments, a SFK inhibitor and another therapeutic can be present in a different formulation when combined in a pharmaceutical composition.

The preparation of a pharmacological composition that contains active ingredients (e.g. a SFK inhibitor) includes various well-known techniques described in the pharmaceutical art.

### 5. TREATMENT REGIMES

In one embodiment, DBA is treated by the methods and compositions of the present disclosure with a SFK inhibitor as disclosed herein.

In some embodiments, the present disclosure relates to treat erythropoietic defects and increase hemoglobin concentration in RPS19 knock-down zebrafish embryos by administering therapeutically effective amount of a SFK inhibitor as disclosed herein.

In some embodiments, a SFK inhibitor can be administered in dose amounts (for a 60 Kg subject) of about 0.01 mg/kg to about 8.5 mg/kg of body weight, about 0.1 mg/kg to about 6.5 mg/kg of body weight, about 0.1 mg/kg to about 4 mg/kg of body weight, about 1 mg/kg to about 4 mg/kg of body weight, about 1.5 mg/kg to about 4 mg/kg of body weight, about 1.5 mg/kg to about 3.25 mg/kg of body weight, inclusive of all ranges and subranges therebetween. In one embodiment, a SFK inhibitor is administered in the dose range of about 1 mg/kg to about 4 mg/kg of body weight or about 1.5 mg/kg to about 3.25 mg/kg of body weight. The dosage may vary within this range depending upon the dosage form employed and the route of administration utilized.

In another embodiment, the dose of Saracatinib is used in an amount of about 0.01 mg/kg to about 8.5 mg/kg of body weight, about 0.1 mg/kg to about 6.5 mg/kg of body weight, about 0.1 mg/kg to about 4 mg/kg of body weight, about 1 mg/kg to about 4 mg/kg of body weight, about 1.5 mg/kg to about 4 mg/kg of body weight, about 1.5 mg/kg to about 3.25 mg/kg of body weight, inclusive of all ranges and subranges therebetween. In one embodiment, the dose of Saracatinib is used in an amount of about 1 mg/kg to about 4 mg/kg of body weight or about 1.5 mg/kg to about 3.25 mg/kg of body weight. In another embodiment, Saracatinib is administered in the dose range of about 1.5 mg/kg to about 3.25 mg/kg body weight of a subject.

In one embodiment, the SFK inhibitor can be administered at a dose of about 0.01 mg/kg of body weight, about 0.1 mg/kg of body weight, about 0.5 mg/kg of body weight, about 0.9 mg/kg of body weight, about 1.0 mg/kg of body weight, about 1.1 mg/kg of body weight, about 1.2 mg/kg of body weight, about 1.3 mg/kg of body weight, about 1.4 mg/kg of body weight, about 1.5 mg/kg of body weight, about 1.6 mg/kg of body weight, about 1.7 mg/kg of body weight, about 1.8 mg/kg of body weight, about 1.9 mg/kg of body weight, about 2.0 mg/kg of body weight, about 2.5 mg/kg of body weight, about 3.0 mg/kg of body weight, about 3.25 mg/kg of body weight, about 3.5 mg/kg of body weight, about 4.0 mg/kg of body weight, about 6.5 mg/kg of body weight, about 8.5 mg/kg of body weight, inclusive of all ranges and subranges therebetween. In another embodiment, the SFK inhibitor can be administered at a dose of about 1.6 mg/kg of body weight. In another embodiment, the SFK inhibitor can be administered at a dose of about 3.25 mg/kg of body weight of a subject.

In one embodiment, the SFK inhibitor can be administered in a unit dose range of about 0.5 mg to about 500 mg, about 5 mg to about 250 mg, and preferably in a unit dose range of about 50 mg to 250 mg, more preferably in a unit dose range of about 100 mg to 250 mg, most preferably in a unit dose range of about 100 mg to 200 mg compounded with an appropriate amount of excipient which may vary from about 5% to 98% by weight of the total composition.

The amount of the SFK inhibitor (Saracatinib) administered for treating the conditions of DBA depends on a variety of factors, which include, but are not limited to age, weight, sex and medical condition of the subject, the type of disease, the severity of the cancer, the route and frequency of administration, and the physical and chemical properties of Saracatinib. Thus, a dosage regimen may vary. In some embodiments, a daily dose of about 5 mg and about 500 mg or about 5 mg to about 250 mg can be used. In one embodiment, a daily dose about 50 mg and about 250 mg can be used. In one embodiment, a daily dose between about 100 mg and about 250 mg can be used. In one embodiment, a daily dose between about 100 mg and about 200 mg may be used. The total dosage per day may be equal to a single dose or the sum of divided doses. The dosage amount can be administered in one to four dosage units per day. The dosages and schedules described hereinbefore may vary depending on the overall condition of the subject. Dose scheduling can be determined by the practitioner using his professional skill and knowledge.

In certain embodiments, the SFK inhibitor can be administered four times a day, thrice a day, twice a day, once a day. In one embodiment, the SFK inhibitor is administered once a day. In other embodiments, the SFK inhibitor can be administered as long as a clinical benefit is observed or until there is a complete response.

In vitro dose of a SFK inhibitor varies from about 10 nM to about 100 μM. The doses (25 μM, 50 μM and 100 μM) of Saracatinib (AZD0530) showed decrease in the Lyn kinase activity with rescue in haemopoietic defect and leads to erythropoiesis in RPS19 knock-down zebrafish embryos.

Effective doses of the pharmaceutical composition comprising a SFK inhibitor as disclosed herein, for the treatment of anemia associated with ribosomal disorders depend upon many different factors, including means of administration, physiological state of the subject, whether the subject is human or an animal, other medications administered, and whether treatment is prophylactic or therapeutic. Depending on the clinical condition of a subject, dosage and frequency of pharmaceutical compositions of the present disclosure can be adjusted accordingly over time. In one embodiment, a SFK inhibitor may be administered, once daily, twice, thrice or four times daily. In one embodiment, a SFK inhibitor is administered once daily.

In therapeutic applications, a relatively high dosage in relatively short intervals is sometimes required until progression of the disease is reduced or terminated, or until the subject shows partial or complete amelioration of symptoms of disease. For example, subjects with DBA can be treated with a SFK inhibitor (e.g. Saracatinib) as disclosed herein at an effective dose in a therapeutic regimen accordingly to regulate the Id2 levels/increase the levels of GATA-1, and then be administered a maintenance dose, e.g., prophylactically. In some embodiments, the SFK inhibitor as disclosed herein can be administered to subjects prior to, concurrently with, or sequentially to treatment with a corticosteroid, a calmodulin inhibitor and/or when the subject is undergoing an adjuvant therapy, such as a blood transfusion and/or bone marrow transplant. In some embodiments, for example, a DBA subject who is selected for other therapeutic procedures or surgeries, such as blood transfusions and/or bone marrow transplant, can be subjected to a treatment with a SFK inhibitor as disclosed herein. In some embodiments, for example, a pharmaceutical composition of the disclosure can be administered prior to, during or after therapeutic procedures.

In some embodiments, the daily dose administered to a subject in a form of a bolus composition comprising a SFK inhibitor can be given in a single dose, in divided doses or in sustained release form effective to obtain the desired results. Second or subsequent administrations can be performed at a dosage which is the same, less than or greater than the initial or previous dose administered to the individual. A second or subsequent administration can be performed during or prior to onset of the disease. It is also within the skill of the art to start doses at levels lower than required to achieve the desired therapeutic effect and to gradually increase the dosage until the desired erythropoiesis is achieved by the decrease in the activity of the Lyn kinase, which in turn regulate the level of Id2 expression and increase in the binding of GATA1 to globin promoter and leads to erythropoiesis.

The methods of the present disclosure also are useful for monitoring a course of treatment being administered to a subject. The methods can be used to monitor both therapeutic treatment on symptomatic subject and prophylactic treatment on asymptomatic subject.

A treatment provided to a subject is considered to be effective if the level of expression of GATA-1 protein in CD34+ cells present in a biological sample obtained from the subject is increased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100%, inclusive of all ranges and subranges therebetween, compared to a reference level, or in the absence of the SFK inhibitor. Said increase in GATA1 level cause increase in erythroid proliferation or increase in the mean corpuscular haemoglobin concentration, which can be assessed from the routine tests known to the person skilled in the art.

A treatment provided to a subject is considered to be effective if the level of activity of the Lyn kinase in CD34+ cells present in a biological sample obtained from the subject is decreased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100%, inclusive of all ranges and subranges therebetween, compared to a reference level, or in the absence of the SFK inhibitor. Said decrease in the Lyn kinase activity cause increase in erythroid proliferation or increase in the mean corpuscular haemoglobin concentration, which can be assessed from the routine tests known to the person skilled in the art.

In one embodiment, the treatment produces at least one therapeutic effect selected from the group comprising of increase in the erythroid proliferation, increase in the mean corpuscular haemoglobin concentration and the like.

### 6. KITS

Another aspect of the present disclosure relates to a kit comprising one or more SFK inhibitor(s) as disclosed herein, and instructions for carrying out a method as disclosed herein.

In some embodiments, a kit can optionally comprise reagents or agents for measuring the level of Id2 protein expression, GATA-1 levels, Lyn kinase activity, increase in the erythroid proliferation, increase in the mean corpuscular haemoglobin concentration in a biological sample from the subject, such as, for example, a blood sample, to identify the efficacy of treatment with the Src family kinase inhibitor(s) as disclosed herein.

In some embodiments, the kit can further comprise instructions for administering a composition comprising a SFK inhibitor to a subject in need thereof, such as a subject with a ribosomal protein disease or disorder, or a subject with DFA, and instructions for doses and the like.

In addition to the above-mentioned component(s), the kit can also include informational material. The informational material can be descriptive, instructional, marketing or other material that relates to the methods described herein and/or the use of the components for the assays, methods and systems described herein.

In some embodiments, the methods and kits comprising a SFK inhibitor as disclosed herein can be performed by a service provider. For example, where an investigator or physician can send the biological sample to a diagnostic laboratory service provider to measure the level of Id2 protein expression in CD34+ cells, and/or the level of GATA1, E2A and/or SCL protein in an erythroid cell population present in the biological sample from the subject, increase in the erythroid proliferation, increase in the mean corpuscular haemoglobin concentration and the like. In one embodiment, after performing such measurements, the service provider can provide the investigator or physician a report of the efficacy of the SFK inhibitor and/or report if the subject is a suitable or amenable to be treated with a SFK inhibitor according to the methods and composition as disclosed herein

In other embodiments, a service provider can provide the investigator with the raw data of the levels of GATA1, E2A and/or SCL protein in an erythroid cell population present in the biological sample from the subject, increase in the erythroid proliferation, increase in the mean corpuscular haemoglobin concentration and leave the analysis to be performed by the investigator or physician. In some embodiments, the report is communicated or sent to the investigator via electronic means, e.g., uploaded on a secure web-site, or sent via e-mail or other electronic communication means. In some embodiments, the investigator can send the samples to the service provider via any means, e.g., via mail, express mail, etc., or alternatively, the service provider can provide a service to collect the samples from the investigator and transport them to the diagnostic laboratories of the service provider. In some embodiments, the investigator can deposit the samples to be analyzed at the location of the service provider diagnostic laboratories.

In other embodiments, the service provider provides a stop-by service, where the service provider send personnel to the laboratories of the investigator and also provides the kits, apparatus, and reagents for performing the assays to measure the level of GATA1, E2A and/or SCL protein in a erythroid cell population present in the biological sample, increase in the erythroid proliferation, increase in the mean corpuscular haemoglobin concentration from the subject as disclosed herein in the investigators laboratories, and analyses the result and provides a report to the investigator for each subject, and leaves the physician to make appropriate recommendations of treatment, and dose to administer the subject with a composition comprising a SFK inhibitor according to the methods as disclosed herein.

It is understood that the foregoing detailed description and the following examples are illustrative only and are not to be taken as limitations upon the scope of the invention. Various changes and modifications to the disclosed embodiments may be made without departing from the spirit and scope of the present disclosure. Further, all patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### 7. SPECIFIC EMBODIMENTS OF THE PRESENT INVENTION ARE AS FOLLOWS

### Embodiment 1

A method of increasing or stimulating erythropoiesis in a subject suffering from anemia associated with a ribosomal disorder comprising administering to said subject an effective amount of a Src family kinase (SFK) inhibitor.

### Embodiment 2

The method as embodied in embodiment 1, wherein the subject has a mutation in ribosomal protein selected from RPL3, RPL5, RPL11, RPL15, RPL26, RPL31, RPL36, RPL35A, RPS7, RPS10, RPS14, RPS17, RPS19, RPS24, RPS26 or RPS29 gene and preferably subject has a mutation in ribosomal protein 19 (RPS19).

### Embodiment 3

The method as embodied in embodiment 1, wherein the anemia associated with ribosomal disorder includes diamond blackfan anemia (DBA).

### Embodiment 4

The method as embodied in embodiment 3, wherein the DBA includes DBA1, DBA2, DBA3, DBA4, DBA5, DBA6, DBA7, DBA8, DBA9, DBA10, DBA11, DBA12 or DBA13; preferably DBA1.

### Embodiment 5

The method as embodied in embodiment 1, wherein the SFK inhibitor inhibits at least one kinase selected from the group comprising of Src, Fyn, Yes, Lck, Lyn, Hck, Fgr, Frk and Blk kinase; preferably Src and/or Lyn kinase.

### Embodiment 6

The method as embodied in embodiment 1, wherein the SFK inhibitor is selected from the group comprising of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably Src family kinase (SFK) inhibitor is Saracatinib [AZD0530], Ponatinib, Bosutinib, Rebastinib, Bafetinib and Dasatinib, more preferably Src family kinase (SFK) inhibitor is Saracatinib [AZD0530].

### Embodiment 7

The method as embodied in embodiment 6, wherein the SFK inhibitor is Saracatinib.

### Embodiment 8

The method as embodied in embodiment 1, wherein the SFK inhibitor regulates the expression level of ID2 protein.

### Embodiment 9

The method as embodied in embodiment 8, wherein the SFK inhibitor further increases or elevates levels of GATA1, E2A and/or SCL; preferably GATA1.

### Embodiment 10

The method as embodied in embodiment 1, wherein the SFK inhibitor is administered in a dose that ranges from about 0.1 mg/kg to about 4 mg/kg, about 1 mg/kg to about 4 mg/kg, about 1.5 mg/kg to about 4 mg/kg, and about 1.5 mg/kg to about 3.25 mg/kg, preferably about 1 mg/kg to about 4 mg/kg and more preferably about 1.5 mg/kg to about 3.25 mg/kg of body weight.

### Embodiment 11

A method of treating a subject suffering from anemia associated with a ribosomal disorder, wherein the method comprises administering to the subject an effective amount of a SFK inhibitor.

### Embodiment 12

The method as embodied in embodiment 11, wherein the subject has mutation in ribosomal protein selected from RPL3, RPL5, RPL11, RPL15, RPL26, RPL31, RPL36, RPL35A, RPS7, RPS10, RPS14, RPS17, RPS19, RPS24, RPS26 or RPS29 gene and preferably subject has a mutation in ribosomal protein 19 (RPS19).

### Embodiment 13

The method as embodied in embodiment 11, wherein the anemia associated with ribosomal disorder includes DBA or inherited erythroblastopenia.

### Embodiment 14

The method as embodied in embodiment 13, wherein DBA includes DBA1, DBA2, DBA3, DBA4, DBA5, DBA6, DBA7, DBA8, DBA9, DBA10, DBA11, DBA12 or DBA13; preferably DBA1.

### Embodiment 15

The method as embodied in embodiment 11, wherein the SFK inhibitor inhibits at least one kinase selected from the group comprising of Src, Fyn, Yes, Lck, Lyn, Hck, Fgr, Frk and Blk kinase; preferably Src and/or Lyn kinase.

### Embodiment 16

The method as embodied in embodiment 11, wherein the SFK inhibitor is selected from the group comprising of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably Src family kinase (SFK) inhibitor is Saracatinib [AZD0530], Ponatinib, Bosutinib, Rebastinib, Bafetinib and Dasatinib, more preferably SFK inhibitor is Saracatinib [AZD0530].

### Embodiment 17

The method as embodied in embodiment 16, wherein the SFK inhibitor is Saracatinib.

### Embodiment 18

The method as embodied in embodiment 11, wherein the SFK inhibitor regulates the expression level of ID2 protein.

### Embodiment 19

The method as embodied in embodiment 18, wherein the SFK inhibitor further increases or elevates levels of GATA1, E2A and/or SCL; preferably GATA1.

### Embodiment 20

The method as embodied in embodiment 11, wherein the SFK inhibitor is administered in a dose that ranges from about 0.1 mg/kg to about 4 mg/kg, about 1 mg/kg to about 4 mg/kg, about 1.5 mg/kg to about 4 mg/kg, and about 1.5 mg/kg to about 3.25 mg/kg, preferably about 1 mg/kg to about 4 mg/kg and more preferably about 1.5 mg/kg to about 3.25 mg/kg of body weight.

### Embodiment 21

A pharmaceutical composition for use in a method for treating a subject suffering from anemia associated with a ribosomal disorder wherein said pharmaceutical composition is administered to the subject comprising an effective amount of a SFK inhibitor.

### Embodiment 22

The pharmaceutical composition for use as embodied in embodiment 21, wherein the subject has a mutation in ribosomal protein selected from RPL3, RPL5, RPL11, RPL15, RPL26, RPL31, RPL36, RPL35A, RPS7, RPS10, RPS14, RPS17, RPS19, RPS24, RPS26 or RPS29 gene and preferably the subject has mutation in ribosomal protein 19 (RPS19).

### Embodiment 23

The pharmaceutical composition for use as embodied in embodiment 21, wherein the anemia associated with ribosomal disorder includes DBA or inherited erythroblastopenia.

### Embodiment 24

The pharmaceutical composition for use as embodied in embodiment 23, wherein DBA includes DBA1, DBA2, DBA3, DBA4, DBA5, DBA6, DBA7, DBA8, DBA9, DBA10, DBA11, DBA12 or DBA13; preferably DBA1.

### Embodiment 25

The pharmaceutical composition for use as embodied in embodiment 21, wherein the SFK inhibitor inhibits at least one kinase selected from the group comprising of Src, Fyn, Yes, Lck, Lyn, Hck, Fgr, Frk and Blk kinase; preferably Src and/or Lyn kinase.

### Embodiment 26

The pharmaceutical composition for use as embodied in embodiment 21, wherein the SFK inhibitor is selected from the group comprising of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably the SFK inhibitor is Saracatinib [AZD0530], Ponatinib, Bosutinib, Rebastinib, Bafetinib and Dasatinib, more preferably the SFK inhibitor is Saracatinib [AZD0530].

### Embodiment 27

The pharmaceutical composition for use as embodied in embodiment 21, wherein the preferred SFK inhibitor is Saracatinib.

### Embodiment 28

The pharmaceutical composition for use as embodied in embodiment 21, wherein the SFK inhibitor regulates the expression level of ID2 protein.

### Embodiment 29

The pharmaceutical composition for use according to embodiment 28, wherein the SFK inhibitor further increases or elevates levels of GATA1, E2A and/or SCL; preferably GATA1.

### Embodiment 30

The pharmaceutical composition for use according to embodiment 21, wherein the pharmaceutical composition comprising the SFK inhibitor in a dose that ranges from about 0.1 mg/kg to about 4 mg/kg, about 1 mg/kg to about 4 mg/kg, about 1.5 mg/kg to about 4 mg/kg, and about 1.5 mg/kg to about 3.25 mg/kg of body weight, preferably about 1 mg/kg to about 4 mg/kg of body weight and more preferably about 1.5 mg/kg to about 3.25 mg/kg of body weight.

### Embodiment 31

A SFK inhibitor for use in the treatment of a subject suffering from anemia associated with ribosomal disorder.

### Embodiment 32

The SFK inhibitor for use as embodied in embodiment 31, wherein the disorder is a result of mutation in RPL3, RPL5, RPL11, RPL15, RPL26, RPL31, RPL36, RPL35A, RPS7, RPS10, RPS14, RPS17, RPS19, RPS24, RPS26 or RPS29 gene and preferably subject has mutation in the ribosomal protein 19 (RPS19).

### Embodiment 33

The SFK inhibitor for use as embodied in embodiment 31, wherein the anemia associated with ribosomal disorder is DBA.

### Embodiment 34

The SFK inhibitor for use as embodied in embodiment 33, wherein the DBA is selected from the group comprising of DBA1, DBA2, DBA3, DBA4, DBA5, DBA6, DBA7, DBA8, DBA9, DBA10, DBA11, DBA12 or DBA13; preferably DBA1.

### Embodiment 35

The SFK inhibitor for use as embodied in embodiment 31, wherein the SFK inhibitor inhibits at least one kinase selected from the group comprising of Src, Fyn, Yes, Lck, Lyn, Hck, Fgr, Frk and Blk kinase; preferably Src and/or Lyn kinase.

### Embodiment 36

The SFK inhibitor for use as embodied in embodiment 31, wherein the SFK inhibitor is selected from the group comprising of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably the SFK inhibitor is Saracatinib [AZD0530], Ponatinib, Bosutinib, Rebastinib, Bafetinib and Dasatinib, more preferably the SFK inhibitor is Saracatinib [AZD0530].

### Embodiment 37

The pharmaceutical composition for use as embodied in embodiment 36, wherein the preferred SFK inhibitor is Saracatinib.

### Embodiment 38

The SFK inhibitor for use as embodied in embodiment 31, wherein the SFK inhibitor regulates the expression level of ID2 protein.

### Embodiment 39

The SFK inhibitor for use as embodied in embodiment 38, wherein the SFK inhibitor increases or elevates levels of GATA1, E2A and/or SCL; preferably GATA1.

### Embodiment 40

The SFK inhibitor for use as embodied in embodiment 31, wherein the SFK inhibitor in a dose ranges from about 0.1 mg/kg to about 4 mg/kg, about 1 mg/kg to about 4 mg/kg, about 1.5 mg/kg to about 4 mg/kg and about 1.5 mg/kg to about 3.25 mg/kg of body weight, preferably about 1 mg/kg to about 4 mg/kg of body weight and more preferably about 1.5 mg/kg to about 3.25 mg/kg of body weight.

### Embodiment 41

A SFK inhibitor for use in increasing or stimulating erythropoiesis in a subject suffering from anemia associated with ribosomal disorder comprising administering to said subject an effective amount of the SFK inhibitor.

### Embodiment 42

The SFK inhibitor for use as in embodiment 41, wherein the disorder is a result of mutation in RPL3, RPL5, RPL11, RPL15, RPL26, RPL31, RPL36, RPL35A, RPS7, RPS10, RPS14, RPS17, RPS19, RPS24, RPS26 or RPS29 gene and preferably subject has mutation in the ribosomal protein 19 (RPS19).

### Embodiment 43

The SFK inhibitor for use as in embodiment 41, wherein the anemia associated with ribosomal disorder is DBA.

### Embodiment 44

The SFK inhibitor for use as embodied in embodiment 43, wherein the DBA is selected from the group comprising of DBA1, DBA2, DBA3, DBA4, DBA5, DBA6, DBA7, DBA8, DBA9, DBA10, DBA11, DBA12 or DBA13; preferably DBA1.

### Embodiment 45

The SFK inhibitor for use as embodied in embodiment 41, wherein the SFK inhibitor inhibits at least one kinase selected from the group comprising of Src, Fyn, Yes, Lck, Lyn, Hck, Fgr, Frk and Blk kinase; preferably Src and/or Lyn kinase.

### Embodiment 46

The SFK inhibitor for use as embodied in embodiment 41, wherein the SFK inhibitor is selected from the group comprising of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably the SFK inhibitor is Saracatinib [AZD0530], Ponatinib, Bosutinib, Rebastinib, Bafetinib and Dasatinib, more preferably the SFK inhibitor is Saracatinib [AZD0530].

### Embodiment 47

The pharmaceutical composition for use as embodied in embodiment 46, wherein the preferred SFK inhibitor is Saracatinib.

### Embodiment 48

The SFK inhibitor for use as embodied in embodiment 41, wherein the SFK inhibitor regulates the expression level of ID2 protein.

### Embodiment 49

The SFK inhibitor for use as embodied in embodiment 48, wherein the SFK inhibitor increases or elevates levels of GATA1, E2A and/or SCL; preferably GATA1.

### Embodiment 50

The SFK inhibitor for use as embodied in embodiment 41, wherein the SFK inhibitor in a dose ranges from about 0.1 mg/kg to about 4 mg/kg, about 1 mg/kg to about 4 mg/kg, about 1.5 mg/kg to about 4 mg/kg and about 1.5 mg/kg to about 3.25 mg/kg of body weight, preferably about 1 mg/kg to about 4 mg/kg of body weight and more preferably about 1.5 mg/kg to about 3.25 mg/kg of body weight.

### Embodiment 51

The methods/compositions according to the preceding claims, wherein the daily dose of the SFK inhibitor may range from about 0.5 mg to about 500 mg, preferably about 5 mg to about 250 mg, more preferably about 50 mg to about 250 mg, even more preferably about 100 mg to about 250 mg and most preferably about 100 mg to about 200 mg.

### Embodiment 52

The methods/compositions according to the preceding claims, wherein the daily dose of Saracatinib may range from about 0.5 mg to about 500 mg, preferably about 5 mg to about 250 mg, more preferably about 50 mg to about 250 mg, even more preferably about 100 mg to about 250 mg and most preferably about 100 mg to about 200 mg.

### Embodiment 53

A kit comprising packaging material and a pharmaceutical composition contained within said packaging material, wherein said pharmaceutical composition comprises the SFK inhibitor, wherein said packaging material comprises a label which indicates that said pharmaceutical composition can be used for treating anemia associated with ribosomal disorder, and wherein said pharmaceutical composition comprises the SFK inhibitor and a pharmaceutically acceptable carrier.

### Embodiment 54

The subject as disclosed in any of the embodiments, wherein the subject is a human.

### 8. EXAMPLE

### Example 1

To evaluate the effect of Saracatinib, a Src kinase inhibitor (Lyn kinase) on improving erythropoiesis in a zebrafish model of DBA using RPS19 morpholino.

Materials: Zebrafish (One celled stage), RPS19 morpholino (1 ng) and standard control morpholino were dissolved in ultrapure water. Saracatinib (Selleckchem; Cat no. 51006) was dissolved in 1% DMSO to achieve 241M, 50 μM and 100 μM concentrations. 0-dianisidine staining solution was made by dissolving 100 mg of 0-dianisidine in 70 mL ethanol, Acetate buffer (49 mL, 0.1 M Sodium acetate and 51 mL, 0.1 M acetic acid), 3% Hydrogen peroxide and ultrapure water; Phosphate buffer saline (PBS), Para formaldehyde (PFA), Tricaine methane sulfonate (ms-222) and Bleach solution (equal parts of 1% Potassium hydroxide and 3% Hydrogen peroxide) were used in the experimental procedures.

Methods:

(i) Control Mo, Egg water represents the zebrafish embryos which were injected with standard control morpholino, and 1% DMSO thriving in egg water,
(ii) RPS19 Mo, Egg water represents wild type zebrafish embryos microinjected with 1 ng of RPS19 morpholino, and 1% DMSO; thriving in egg water, and
(iii) RPS19 Mo+Saracatinib injected 1 cell stage and aqueous exposure represents zebrafish embryos which were microinjected with RPS19 morpholino and where Saracatinib was also microinjected at 1-cell stage along with adding Saracatinib in the aqueous phase or egg water.

The wild type zebrafish with egg water were placed in separate wells and were microinjected with standard control morpholino and 1% DMSO, which acted as normal control (Control Mo, Egg water). The wild type zebrafish eggs were injected with 1 ng of RPS19 morpholino and 1% DMSO at 1-cell stage (RPS19 Mo, Egg water), and were incubated at 28.5° C. for 96 hours. Saracatinib was microinjected in the zebra fish at one cell stage and also exposed in the media (aqueous exposure) to achieve optimal concentrations of 25 μM, 50 μM and 100 μM (RPS19 Mo+Saracatinib injected 1 cell stage and aqueous exposure). The experiment was carried out in the 6-well plates for control (Control Mo, Egg water); morpholinos group (RPS19 Mo, Egg water) and Saracatinib treatment group (RPS19 Mo+Saracatinib injected 1 cell stage and aqueous exposure) respectively. The incubation of the zebrafish in various groups was carried out up to 96 hours at 28.5° C. and the embryos were visually observed for any phenotypic changes or mortality. The embryos were euthanized at the end of the study by using tricaine methane sulfonate (ms-222), and fixed in 4% PFA for 2 hours at room temperature. After fixing and washing the embryos thoroughly (3 times) in 1×PBS, o-dianisidine staining was carried out for 30 minutes at room temperature in the dark to assess haemoglobin concentration. Embryos were then washed 3 times in 1×PBS and incubated in bleach solution for 20 minutes at room temperature. Further washing of the embryos were also carried out in 1×PBS to avoid any unnecessary staining. Quantitation of o-dianisidine staining in zebrafish embryos.

Haemoglobin quantification/total amount of the o-dianisidine staining on total embryo was calculated by using Image J software. The total embryo area and the intensity was noted by the software, by manually tracing the exact area of the embryo. The ratios of the intensity and the total embryo area was thus used to calculate the intensity per unit area which is then compared across groups (Control Mo, Egg water; RPS19 Mo, Egg water and RPS19 Mo+Saracatinib injected 1 cell stage and aqueous exposure). Percent intensity of staining of embryos was considered to be proportional to the haemoglobin concentration in each embryo and thus % improvement or recovery of erythropoiesis by administration of Saracatinib was further calculated.

www.imagej.net/

$$\% improvement of erythropoiesis is calculated \text{as:}$$

$$\frac{\begin{matrix}[ ( RPS 19 Mo + Saracatinib injected 1 cell stage \& \\ aqueous exposure ) - \\ ( RPS 19 Mo , Egg water ) ]\end{matrix}}{[ ( Conrol Mo , Egg water ) - ( RPS 19 Mo , Egg water ) ]} \times 100$$

Results:

| | Area of | Intensity | Ratio of staining | % intensity | % improvement |
|---|---|---|---|---|---|
| 96 hpf | staining | of staining | (Intensity/unit area) | of staining | of erythropoiesis |
| 25 μM | | | | | |
| Control Mo, Egg water | 3011 | 130.54 | 0.04335436732 | 4.335436732 | 10.49% |
| RPS 19 Mo, Egg water | 4440 | 108.71 | 0.02448423423 | 2.448423423 | |
| RPS19 Mo + Saracatinib | 5220 | 138.143 | 0.02646417625 | 2.646417625 | |
| injected 1 cell stage & | | | | | |
| aqueous exposure | | | | | |
| 50 μM | | | | | |
| Control Mo, Egg water | 2292 | 127.417 | 0.05559205934 | 5.559205934 | 36.75% |
| RPS 19 Mo, Egg water | 5440 | 130.249 | 0.02394283088 | 2.394283088 | |
| RPS19 Mo + Saracatinib | 3831 | 136.283 | 0.03557374054 | 3.557374054 | |
| injected 1 cell stage & | | | | | |
| aqueous exposure | | | | | |
| 100 μM | | | | | |
| Control Mo, Egg water | 1452 | 92.345 | 0.06359848485 | 6.359848485 | 19.82% |
| RPS 19 Mo, Egg water | 5440 | 130.249 | 0.02394283088 | 2.394283088 | |
| RPS19 Mo + Saracatinib | 3264 | 103.807 | 0.03180361520 | 3.180361520 | |
| injected 1 cell stage & | | | | | |
| aqueous exposure | | | | | |

Saracatinib administration led to an improvement in erythropoiesis in a zebrafish model of diamond blackfan anemia. RPS19 morpholinos at 1 ng concentration showed significant decrease in the haemoglobin concentration, as observed by the decrease in the o-dianisidine staining of the embryos as compared to the wild type controls at 96 hours post fertilization (hpf). This also signified the deficiency of hematopoietic progenitors, in particular, the erythroid lineage cells produced by the knockdown to cause erythropoietic defect in the zebrafish, a hallmark of the DBA syndrome.

Saracatinib at varying concentrations (25 μM, 50 μM, and 100 μM) showed a dose related improvement in erythropoiesis as observed by o-dianisidine staining as compared to the RPS19 injected morpholinos at 96 hours post fertilization.

In conclusions, the experiment clearly demonstrated the effect of RPS19 knockdown in producing the erythropoietic defect and low haemoglobin concentrations in the zebrafish, which is a hallmark of DBA in humans. Saracatinib also showed an improvement in the erythropoietic defect and increased haemoglobin concentration as compared to the zebrafish treated with RPS19 morpholino, showing clear therapeutic utility of Saracatinib (SFK inhibitor) in reversing the DBA phenotype.

### 9. REFERENCES

Oliver Gautschi et al., Regulation of Id1 expression by SRC: implications for targeting of the bone morphogenetic protein pathway in Cancer; The Journal of Cancer Research; Vol. 68 (7), 2008, 2250-2258.
Min-Ying Zhang et al. Expression of SCL is normal in transfusion-dependent diamond blackfan anemia but other bHLH proteins are deficient; Blood 1997 (American Society of Hematology); Vol. 90 (5), Page No. 2068-2074.
Wonil Kim et al., Gfi-1 regulates the erythroid transcription factor network through Id2 repression in murine hematopoietic progenitor cells; Blood, 2014; Vol. 124 (10), Page No. 1586-1596.
Huajie Li et al., Repression of Id2 expression by Gfi-1 is required for B-cell and myeloid development; Blood, 2010; Vol. 116 (7), Page No. 1060-1069.
Slavova-Azmanova et al; Gain-of-function Lyn induces anemia: appropriate Lyn activity is essential for normal erythropoiesis and Epo receptor signaling; Blood.; 2013; 122(2):262-71.
US Publication No. 20150265627 published on 24 Sep. 2015.
Soriano et. al, Targeted disruption of the c-src proto-oncogene leads to osteoporosis in mice; Cell, 1991, 64(4), 693-702.
Boyce et. al, Requirement of pp60 c-src expression for osteoclasts to form ruffled borders and resorb bone in mice, The Journal of Clinical Investigation; 1992, 90(4), 1622-1627.
Yoneda et al, Mycotrienins: A new class of potent inhibitors of osteoclastic bone resorption; The Journal of Clinical Investigation; 1993, 91, 2791-2795.
Missbach et al, A novel inhibitor of the tyrosine kinase Src suppresses phosphorylation of its major cellular substrates and reduces bone resorption in vitro and in rodent models in vivo; Bone, 1999, 24(5), 437-449.
Owens et al, The catalytic activity of the Src family kinases is required to disrupt cadherin-dependent cell-cell contacts; Molecular Biology of Cell; 2000; 11(1), 51-64.
Klinghoffer et al, Src family kinases are required for integrin but not PDGFR signal transduction; EMBO Journal, 1999, 18(9), 2459-2471.

### INCORPORATION BY REFERENCE

All references, articles, publications, patents, patent publications, and patent applications cited herein are incorporated by reference in their entireties for all purposes.

1. A method of increasing or stimulating erythropoiesis in a subject suffering from anemia associated with a ribosomal disorder comprising administering to said subject an effective amount of a Src family kinase (SFK) inhibitor.

2. The method of claim 1, wherein the subject has a mutation in ribosomal protein selected from the group consisting of RPL3, RPL5, RPL11, RPL15, RPL26, RPL31, RPL36, RPL35A, RPS7, RPS10, RPS14, RPS17, RPS19, RPS24, RPS26, and RPS29 gene, and preferably the subject has a mutation in RPS19.

3. The method of claim 1, wherein the anemia associated with the ribosomal disorder includes diamond blackfan anemia (DBA).

4. The method of claim 3, wherein the DBA includes DBA1, DBA2, DBA3, DBA4, DBA5, DBA6, DBA7, DBA8, DBA9, DBA10, DBA11, DBA12 or DBA13; preferably DBA1.

5. The method of claim 1, wherein the SFK inhibitor inhibits at least one kinase selected from the group consisting of Src, Fyn, Yes, Lck, Lyn, Hck, Fgr, Frk and Blk kinase; preferably Src and/or Lyn kinase.

6. The method aof claim 1, wherein the SFK inhibitor is selected from the group consisting of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably the SFK inhibitor is Saracatinib [AZD0530], Ponatinib, Bosutinib, Rebastinib, Bafetinib and Dasatinib, more preferably the SFK inhibitor is Saracatinib [AZD0530].

7. The method of claim 6, wherein the SFK inhibitor is Saracatinib.

8. The method of claim 1, wherein the SFK inhibitor regulates the expression level of ID2 protein.

9. The method of claim 8, wherein the SFK inhibitor further increases or elevates levels of GATA1, E2A and/or SCL; preferably GATA1.

10. The method of claim 1, wherein the SFK inhibitor is administered in a dose ranging from about 0.1 mg/kg to about 4 mg/kg, about 1 mg/kg to about 4 mg/kg, about 1.5 mg/kg to about 4 mg/kg, about 1.5 mg/kg to about 3.25 mg/kg of body weight, preferably about 1 mg/kg to about 4 mg/kg of body weight, more preferably about 1.5 mg/kg to about 3.25 mg/kg of body weight.

11. A method of treating a subject suffering from anemia associated with a ribosomal disorder, wherein the method comprises administering to the subject an effective amount of a SFK inhibitor.

12. The method of claim 11, wherein the subject has mutation in ribosomal protein selected from the group consisting of RPL3, RPL5, RPL11, RPL15, RPL26, RPL31, RPL36, RPL35A, RPS7, RPS10, RPS14, RPS17, RPS19, RPS24, RPS26, and RPS29 gene, and preferably the subject has a mutation in RPS19.

13. The method of claim 11, wherein the anemia associated with the ribosomal disorder includes DBA.

14. The method of claim 13, wherein DBA comprises DBA1, DBA2, DBA3, DBA4, DBA5, DBA6, DBA7, DBA8, DBA9, DBA10, DBA11, DBA12 or DBA13; preferably DBA1.

15. The method of claim 11, wherein the SFK inhibitor inhibits at least one kinase selected from the group consisting of Src, Fyn, Yes, Lck, Lyn, Hck, Fgr, Frk and Blk kinase; preferably Src and/or Lyn kinase.

16. The method of claim 11, wherein the SFK inhibitor is selected from the group consisting of Saracatinib [AZD0530], Quercetin, Bosutinib, Dasatinib, Rebastinib, Staurosporine, A419259, KX2-391, PP1, PP2, Ponatinib, SU6657, Bafetinib, USC15A, Genistein, CGP77675, CGP76030, Naktide, Piceatannol, Lavendustin A, SU6656, AP22408, AP23451, AP23588, AP23846, AP23848, AP23464, AP23994, PD180970, SKS-927, XL-228, JNJ-26483327, CT5269, AZM559756, AP23451, PD173955, Radicicol R2146, Geldanamycin, Herbimycin A, AZD0424, NS-018 and pD166326; preferably the SFK inhibitor is Saracatinib [AZD0530], Ponatinib, Bosutinib, Rebastinib, Bafetinib and Dasatinib, more preferably the SFK inhibitor is Saracatinib [AZD0530].

17. The method of claim 15, wherein the SFK inhibitor is Saracatinib.

18. The method of claim 11, wherein the SFK inhibitor regulates the expression level of ID2 protein.

19. The method of claim 18, wherein the SFK inhibitor further increases or elevates levels of GATA1, E2A and/or SCL; preferably GATA1.

20. The method of claim 11, wherein the SFK inhibitor is administered in a dose ranging from about 0.1 mg/kg to about 4 mg/kg, about 1 mg/kg to about 4 mg/kg, about 1.5 mg/kg to about 4 mg/kg, about 1.5 mg/kg to about 3.25 mg/kg of body weight, preferably about 1 mg/kg to about 4 mg/kg of body weight, more preferably about 1.5 mg/kg to about 3.25 mg/kg of body weight.
